(19) United States
(12) Patent Application Publication
(10) Pub. No.: US 2013/0058883 A1
(43) Pub. Date: Mar. 7, 2013

(54) COMPOSITION FOR MAKING UP AND/OR CARING FOR KERATINOUS FIBERS AND PRESENTING IMPROVED STAYING-POWER PROPERTIES

(71) Applicants: Guillaume Kergosien, Chaville (FR); Maitena Leuridan, Brie Comte Robert (FR); Nathalie Jager-Lezer, Verrieres-Le-Buisson (FR)

(72) Inventors: Guillaume Kergosien, Chaville (FR); Maitena Leuridan, Brie Comte Robert (FR); Nathalie Jager-Lezer, Verrieres-Le-Buisson (FR)

(73) Assignee: L'OREAL, Paris (FR)

(21) Appl. No.: 13/698,207

(22) PCT Filed: May 17, 2011
(86) PCT No.: PCT/IB11/52158

§ 371 Date: Nov. 15, 2012

Related U.S. Application Data
(60) Provisional application No. 61/367,547, filed on Jul. 26, 2010.

(30) Foreign Application Priority Data
May 17, 2010 (FR) 1053799

(51) Int. Cl. A61K 8/92; A61Q 1/10; A61Q 5/00

(52) U.S. Cl. 424/70.7; 424/70.1

(57) ABSTRACT

A cosmetic composition for making up and/or caring for keratinous fibers. The composition includes at least: an oily phase formed at least of isododecane, and at least 4% by weight, relative to the total weight of the composition, of at least one oil of volatility that is less than that of isododecane; at least 9% by weight, relative to the total weight of the composition, of active material including at least one liposoluble film-forming polymer; and a particulate phase containing at least one compound selected from pigments, nacres, fillers, and waxes.

The present invention provides cosmetic compositions for making up and/or caring for keratinous fibers.

It also provides a method of making up and/or caring for keratinous fibers.

The compositions and methods of the invention are more particularly for human keratinous fibers, such as the eyelashes, the eyebrows, and the hair, including false eyelashes and hair pieces.

In particular, the compositions of the invention may be makeup compositions, makeup bases, compositions for applying on makeup, also known as “top-coats”, or even cosmetic treatment compositions for treating keratinous fibers. More generally, the invention relates to a mascara.

One of the problems generally encountered after coating keratinous fibers, e.g. the eyelashes, is that the resulting film tends to disintegrate over time. Grains detach from the applied film and are deposited in the vicinity of said keratinous fibers, leaving, in the case of mascaras, unattractive traces, in particular around the eye.

An additional problem is that the film when weakened in this way presents less resistance to friction or rubbing, in particular on coming into contact with the fingers, or even with water while bathing or showering, for example. The coating is thus no longer sufficiently resistant and presents poor staying power.

In order to solve this problem, it is already known to use tackifying compounds that, by means of their tackifying properties, improve the staying-power properties of compositions that contain them.

Nevertheless, this tackifying character presents the drawback of causing keratinous fibers to stick together, which, for obvious reasons, is unacceptable to the user.

An object of the present invention is to propose a composition that is capable of forming a film deposit presenting staying power and good resistance to friction, without presenting the above-mentioned drawbacks, and in particular manifesting an adhesive character that is significantly less than that observed in the above-described conventional formulae.

The inventors have observed that such a result could be achieved effectively with a composition comprising at least an oily phase, a liposoluble film-forming polymer, and a particulate phase, in particular in the presence of a certain content of an oil of volatility that is less than the volatility of isododecane and of said liposoluble film-forming polymer.

Thus, first exemplary embodiments of the present invention provide a cosmetic composition for making up and/or caring for keratinous fibers, said composition comprising at least:

a) an oily phase formed at least of:
isododecane; and
at least 4% by weight, relative to the total weight of the composition, of at least one oil of volatility that is less than that of isododecane;

b) at least 9% by weight, relative to the total weight of the composition, of active material of at least one liposoluble film-forming polymer; and

c) a particulate phase containing at least one compound selected from pigments, nacres, fillers, and waxes.

More particularly, the present invention provides a cosmetic composition for making up and/or caring for keratinous fibers, said composition comprising at least:

a) an oily phase formed at least of:
isododecane; and
at least 4% by weight, relative to the total weight of the composition, of at least one oil of volatility that is less than that of isododecane;

b) at least 9% by weight, relative to the total weight of the composition, of active material of at least one liposoluble film-forming polymer; and

c) a particulate phase containing at least one compound selected from pigments, nacres, fillers, and waxes, and representing at least 15% by weight of the total weight of the composition;

said composition including at least 20% by weight of isododecane relative to its total weight; the content by weight of the oil of volatility that is less than that of isododecane being less than the content by weight of the particulate phase.

The compositions of the invention are easy to apply and they coat keratinous fibers well, in particular the eyelashes.

The deposited film is natural, comfortable, and presents good staying power.

In an advantageous embodiment, the composition of the invention includes less than 5% by weight of rosin ester, preferably less than 2% by weight, or is even exempt of rosin ester, in particular of glycerol and pentaerythritol ester(s).

In exemplary embodiments, the composition of the invention is anhydrous.

The term “anhydrous composition” means a composition containing less than 2% by weight of water, or even less than 0.5% by weight of water, and in particular that is exempt of water.

Where appropriate, equally small quantities of water may in particular be contributed by ingredients of the composition that may contain residual quantities of water.

The compositions of the invention advantageously present an adhesive value that is less than or equal to 0 newtons (N), e.g. lying in the range −0.3 N to 0 N, preferably in the range −0.2 N to 0 N, more preferably in the range −0.15 N to 0 N, the limits being included in the ranges.

In particular, the adhesive character of a composition may be evaluated by means of the protocol described below.

Initially, the composition is coated onto a glass plate at a thickness, before drying, of 100 micrometers (μm), then allowed to dry for 24 hours at 20° C.

By means of a texture analyzer (TAXT2i), the adhesive strength of a prepared composition sample is then evaluated by applying a double-sided adhesive onto a plate at 20° C., and by positioning the glass plate on the double-sided adhesive so as to hold it in place.

The measurement parameters are as follows:
Tack probe: stainless steel cylinder (reference SMS P/5), of diameter 5 millimeters (mm);
Speed prior to compression=0.1 millimeters/second (mm/s);
Compression force (compression step)=0.01 megapascals (MPa) (for 20 seconds); and
Speed after compression (traction step)=0.1 mm/s.

The maximum release force is thus evaluated (value expressed negatively) during the traction step on six samples. The values are expressed in newtons. A average value is calculated on all six samples.

The measurements are carried in an environment at 20° C., with 30% relative humidity (RH).

In other exemplary embodiments the present invention also relates to a method of making up and/or caring for keratinous fibers, said method comprising at least applying at least one composition as defined above on said keratinous fibers.

### Oily Phase

The composition of the invention includes at least one oily phase formed of oils (i.e. of fats that are liquid at ambient temperature (25° C.) and at atmospheric pressure (760 millimeters of mercury (mmHg), or 105 pascals (Pa))) or of organic solvents.

The oily phase is formed at least of isododecane and of at least one oil of volatility that is less than that of isododecane.

### Isododecane

The composition of the invention may include at least 25% by weight, e.g. at least 30% by weight relative to the total weight of the composition.

According to the invention, the content by weight of isododecane in the composition is greater than the content by weight of the oil of volatility that is less than that of isododecane.

Oil of Volatility that is Less than that of Isododecane

In the meaning of the invention, the term “oil of volatility that is less than that of isododecane” means oils presenting a rate of evaporation that is less than 0.8 micrograms per square centimeter per minute (mg/cm²/min), at ambient temperature (25° C.) and at atmospheric pressure (760 mmHg).

Preferably, the oil may present a rate of evaporation that is less than 0.1 mg/cm²/min, preferably less than 0.01 mg/cm²/min.

In particular, the rate of evaporation of an oil of the invention may be evaluated by means of the protocol described in WO 06/013413, and more particularly by means of the protocol described below.

15 grams (g) of oil are introduced into a crystallizer (diameter: 7 centimeters (cm)) placed on a balance in a temperature-regulated (25° C.) and humidity-regulated (relative humidity 50%) chamber of approximately 0.3 cubic meters (m³).

The liquid is allowed to evaporate freely without stirring, with ventilation being provided by a fan (PAPST-MOTOREN, reference 8550 N, turning at 2700 revolutions per minute (rpm) disposed in a vertical position above the crystallizer containing the oil, the blades being directed towards the crystallizer, at a distance of 20 cm relative to the bottom of the crystallizer.

At regular intervals, the mass of oil remaining in the crystallizer is measured.

The evaporation profile of the solvent is thus obtained by plotting the curve showing the quantity of substance that has evaporated (in mg/cm²) as a function of time (in min).

Then, the rate of evaporation is calculated as corresponding to the tangent at the origin of the resulting curve. The rates of evaporation are expressed in mg of oil evaporated per unit surface area (cm²) and per unit time (minute).

The oil of volatility that is less than that of isododecane may be selected from hydrocarbon oils, silicone oils, and fluorinated oils.

Preferably, it is a hydrocarbon oil, e.g. as described below.
Hydrocarbon oils

The hydrocarbon oil may be selected from linear or branched volatile hydrocarbon oils having at least 13 carbon atoms, in particular having 13 to 16 carbon atoms. It may also be a non-volatile hydrocarbon oil.

Examples of particular hydrocarbon oils containing 13 to 16 carbon atoms that may be mentioned are C₁₃-C₁₆ branched alkanes such as C₁₃-C₁₆ iso-alkanes (also termed isoparaffins). Preferably, the volatile hydrocarbon oil containing 13 to 16 carbon atoms is isohexadecane.

Examples of linear alkanes that are suitable for the invention and that may be mentioned are n-tridecane (C₁₃), n-tetradecane (C₁₄) (in particular sold by Sasol under the reference PARAFOL 14-97), and mixtures thereof.

Examples of hydrocarbon oils of volatility that is less than that of isododecane that may be used are: paraffin oil (or vaseline), squalane, hydrogenated polyisobutylene (Parleam oil), perhydrosqualene, mink oil, turtle oil, soybean oil, sweet almond oil, calophyllum oil, palm oil, grapeseed oil, sesame seed, corn oil, arara oil, rapeseed oil, sunflower oil, cottonseed oil, apricot oil, castor oil, avocado oil, jojoba oil, olive oil, or cereal-germ oil; esters of lanolic acid, oleic acid, lauryl acid, stearic acid; fatty esters, in particular C₁₂-C₃₆ fatty esters, such as isopropyl myristate, isopropyl palmitate, butyl stearate, hexyl laurate, diisopropyl adipate, isononyl isononanoate, 2-ethyl-hexyl palmitate, 2-hexyl-decyl laurate, 2-octyl-decyl palmitate, 2-octyl-dodecyl myristate or lactate, di(2-ethyl hexyl) succinate, diisostearyl malate, glycerine or diglycerine triisostearate; behenic acid, oleic acid, linoleic acid, linolenic acid, or isostearic acid; higher fatty alcohols, in particular at C₁₆-C₂₂ higher fatty alcohols, such as cetanol, oleic alcohol, linoleic or linolenic alcohol, octyl dodecanol or isostearic alcohol; and mixtures thereof. Preferably, the non-volatile hydrocarbon oil is hydrogenated polyisobutylene.
Silicone oils

In a variant or in addition, the composition of the invention may include at least one volatile silicone oil that is compatible with cosmetic use and of volatility that is less than that of isododecane.

The term “silicone oil” means an oil containing at least one silicon atom, and in particular containing Si—O groups.

In an embodiment, said composition includes less than 10% by weight of non-volatile silicone oil relative to the total weight of the composition, better less than 5% by weight, or is even exempt of silicone oil.

Examples of volatile silicone oils that may be mentioned are cyclic polysiloxanes, linear polysiloxanes, and mixtures thereof.

Examples of linear volatile polysiloxanes that may be mentioned are hexamethyldisiloxane, octamethyl-trisiloxane, decamethyltetrasiloxane, tetradecamethyl-hexasiloxane, and hexadecamethylheptasiloxane.

Examples of cyclic volatile polysiloxanes that may be mentioned are hexamethylcyclotrisiloxane, octamethyl-cylotetrasiloxane, decamethylcyclopentasiloxane, and dodecamethylcyclohexasiloxane.

In an embodiment, the composition of the invention includes less than 5% by weight of cyclic polydimethyl-siloxane, in particular of decamethylcyclopentasiloxane, e.g. less than 2% by weight, or is even exempt of cyclic polydimethylsiloxane.
Fluorinated oils

In a variant or in addition, the composition of the invention may include at least one volatile fluorinated oil of volatility that is less than that of isododecane.

The term “fluorinated oil” means an oil containing at least one fluorine atom.

Examples of volatile fluorinated oils that may be mentioned are nonafluoromethoxybutane or perfluoromethyl-cyclopentane, and mixtures thereof.

Particular non-volatile fluorinated oils that may be used in the invention are fluorosilicone oils, fluorinated polyethers, and fluorinated silicones such as those described in document EP-A-0 847 752.

In a composition of the invention, the oil of volatility that is less than that of the isododecane is present at a content that is greater than or equal to 4% by weight relative to the total weight of the composition.

In particular, it may be present at a content that is greater than or equal to 5% by weight relative to the total weight of said composition, e.g. lying in the range 5% to 90% by weight, e.g. greater than or equal to 10% by weight, e.g. lying in the range 10% to 50% by weight;

In exemplary embodiments, the oil of volatility that is less than that of isododecane is selected from hydrogenated polyisobutylene, isohexadecane, and mixtures thereof.

Preferably, the oil of volatility that is less than that of isododecane is isohexadecane.

In an embodiment, the composition of the invention comprises in the oily phase, in addition to isododecane and an oil of volatility that is less than that of isododecane as defined previously, a second oil of volatility that is less than that of isododecane.

This second oil of volatility that is less than that of isododecane may preferably be a non-volatile oil.

Preferably, the non volatile oil is a non-volatile hydrocarbon oil and is more particularly hydrogenated polyisobutylene.

In particular, the composition of the invention includes preferably less than 10% by weight of second oil of volatility that is less than that of isododecane, in particular less than 5% by weight of the total weight of the composition, or is even exempt of this second oil.

Accordingly, the composition of the invention includes preferably less than 10% by weight of hydrogenated polyisobutylene, in particular less than 5% by weight of the total weight of the composition, or is even exempt of hydrogenated polyisobutylene.

In an embodiment, the composition of the invention includes at least an oily phase formed of isododecane, isohexodecane and hydrogenated polyisobutylene.

In variant exemplary embodiments, isododecane and oil of volatility that is less than that of isododecane are present in a weight ratio [weight of isododecane divided by weight of the oil of volatility that is less than that of isododecane] at least equal to 1.5, preferably greater than 2.

In an embodiment, the present invention provides a cosmetic composition for making up and/or caring for keratinous fibers, said composition comprising at least:
a) an oily phase formed at least of
   isododecane; and
   at least 4% by, relative to the total weight of the composition, of a combination of isohexadecane and hydrogenated polyisobutylene;
b) at least 9% by weight, relative to the total weight of the composition, of active material of at least one liposoluble film-forming polymer; and
c) a particulate phase containing a least one compound selected from pigments, nacres, fillers, and waxes, and representing at least 15% by weight of the total weight of the composition;
   said composition including at least 20% by weight of isododecane relative to its total weight;
   the content by weight of the combination of isohexodecane and hydrogenated polyisobutylene being less than the content by weight of the particulate phase.

### Liposoluble Film-Forming Polymer

As indicated above, the composition of the invention includes at least one liposoluble film-forming polymer, i.e. soluble in an oily phase including organic oils or solvents such as those above-described.

Advantageously, the liposoluble polymer of the invention is a hydrocarbon “tackifier” resin. In particular, such resins are described in the Handbook of Pressure Sensitive Adhesive, edited by Donatas Satas, 3rd ed., 1989, pp. 609-619.

Said lipophilic polymer is preferably a hydrocarbon polymer, such as an olefin polymer or copolymer or an aromatic hydrocarbon monomer polymer or copolymer, preferably an indene hydrocarbon resin, e.g. as described below. Said polymer may be hydrogenated, partially hydrogenated, or non-hydrogenated.

Preferably, said polymer presents a softening point that is less than 120° C., preferably less than 110° C.

In the context of the present invention, the softening point is measured using the ring-and-ball method in accordance with the ASTM D36 Standard. To do this, use is made of an automatic NBA 440 tester available from Normalab. The fluid used for the measurement is glycerine.

Hydrocarbon resins of the invention are selected from polymers that may, depending on the type of monomer that they contain, be classified as:
indene hydrocarbon resins such as the resins derived from the polymerization of indene monomer in the greater proportion, and of a monomer selected from styrene, methylindene, methylstyrene, and mixtures thereof in the lesser proportion. These resins may possibly be hydrogenated. They may present a molecular weight lying in the range 290 grams per mole (g/mol) to 1150 g/mol.

Examples of indene resins that may be mentioned are those sold under the reference NORSOLENE S95, NORSOLENE S105, NORSOLENE 5115 by the Supplier Cray Valley, or hydrogenated indene/methylstyrene/styrene copolymers sold under the name “REGALITE” by the supplier Eastman Chemical, in particular REGALITE C6100, REGALITE C6100L, REGALITE R1090, REGALITE R1100, REGALITE R7100, REGALITE R9100, REGALITE S1100, REGALITE S5100, or under the name ARKON P-90, ARKON P-100, ARKON P-115, ARKON M-90, ARKON M-100, ARKON M-115 by the supplier Arakawa.
aliphatic pentadiene resins such as that derived from the polymerization mainly of 1,3-pentanediene (trans or cis piperylene) and of a minor monomer selected from isoprene, butene, 2-methyl-2-butene, pentene, 1,4-pentadiene and mixtures thereof. These resins may present a molecular weight lying in the range 1000 g/mol to 2500 g/mol.

By way of example, such 1,3-pentadiene resins are sold under the references PICCOTAC 95 by the supplier Eastman Chemical, ESCOREZ 1102, ESCOREZ 1304, ESCOREZ 1310LC, ESCOREZ 1315 by the supplier Exxon Chemicals, WINGTACK 95 by the supplier Cray Valley;
mixed pentadiene and indene resins that are derived from the polymerization of a mixture of pentadiene and indene monomers such as those described above, such as for example the resins sold under the reference ESCOREZ 2101, ESCOREZ 2105, ESCOREZ 2173, ESCOREZ 2184, ESCOREZ 2203LC, ESCOREZ 2394, ESCOREZ 2510 by the supplier Exxon Chemicals, NORSOLENE A 100 by the supplier Cray Valley, the resins sold under the reference WINGTACK 86, WINGTACK EXTRA and WINGTACK PLUS by the supplier Cray Valley;
polycyclopentadienes such as those having the reference KOBOGUARD 5400 sold by the supplier KOBO.
diene resins from cyclopentadiene dimers, such as those derived from the polymerization of a first monomer selected from indene and styrene, and a second monomer selected from dimers of cyclopentadiene such as dicyclopentadiene, methyldicyclopentadiene, other dimers of pentadiene, and mixtures thereof. These resins generally present a molecular weight lying in the range 500 g/mol to 800 g/mol, such as for example those sold under the reference ESCOREZ 5380, ESCOREZ 5300, ESCOREZ 5400, ESCOREZ 5415, ESCOREZ 5490, ESCOREZ 5600, ESCOREZ 5615, ESCOREZ 5690, by the supplier Exxon Mobil Chem., and the resins SUKOREZ SU-90, SUKOREZ SU-100, SUKOREZ SU-110, SUKOREZ SU-100S, SUKOREZ SU-200, SUKOREZ SU-210, SUKOREZ SU-490, SUKOREZ SU-400, by the supplier Kolon;
diene resins from isoprene dimers such as the terpene resins derived from the polymerization of at least one monomer selected from α-pinene, β-pinene, limonene, styrene, and mixtures thereof. These resins may present a molecular weight lying in the range 300 g/mol to 2000 g/mol. By way of example, such resins are sold under the name PICCOLYTE A115 by the supplier Hercules, ZONAREZ 7100 or ZONATAC 105 LITE by the supplier ARIZONA Chem.

Mention may also be made of hydrogenated resins derived mainly from the polymerization of pentadiene such as those sold under the name EASTOTAC H-100E, EASTOTAC H-115E, EASTOTAC C-100L, EASTOTAC C-115L, EASTOTAC H-100L, EASTOTAC H-115L, EASTOTAC C-100R, EASTOTAC C-115R, EASTOTAC H-100R, EASTOTAC H-115R, EASTOTAC C-100W, EASTOTAC C-115W, EASTOTAC H-100W, EASTOTAC H-115W, by the supplier Eastman Chemical Co.

In particular exemplary embodiments, the resin is selected from indene hydrocarbon resins sold under the name NORSOLENE S95, NORSOLENE 5105, NORSOLENE 5115 by the supplier Cray Valley, under the name “REGALITE” by the supplier Eastman Chemical, in particular REGALITE C6100, REGALITE C6100L, REGALITE R1090, REGALITE R1100, REGALITE R7100, REGALITE R9100, REGALITE S1100, REGALITE S5100, or under the name ARKON P-90, ARKON P-100, ARKON P-115, ARKON M-90, ARKON M-100, ARKON M-115 by the supplier Arakawa.

Examples of liposoluble polymers that may also be mentioned are vinyl ester copolymers (the vinyl group being directly bonded to the oxygen atom of the ester group and the vinyl ester having a linear or branched saturated hydrocarbon radical containing 1 to 19 carbon atoms, bonded to the carbonyl of the ester group) and at least one other monomer that may be a vinyl ester (different from the already present vinyl ester), an α-olefin (containing 8 to 28 carbon atoms), an alkylvinylether (in which the alkyl group contains 2 to 18 carbon atoms), or an allyl or methallyl ester (containing a saturated, linear or branched hydrocarbon radical containing 1 to 19 carbon atoms, bonded to the carbonyl of the ester group).

These copolymers may be cross-linked with the aid of cross-linking agents that may either be of the vinyl type or of the allyl or methallyl type, such as tetraallyl-oxyethane, divinylbenzene, divinyl octanedioate, divinyl dodecanedioate, or divinyl octadecanedioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/octadecenel, vinyl acetate/dodecenel, vinyl stearate/ethylvinylether, vinyl propionate/cetyl vinyl ether, vinyl stearate/allyl acetate, 2,2-dimethyl vinyl octanoate/vinyl laurate, 2,2-dimethyl allyl pentanoate/vinyl laurate, dimethyl vinyl propionate/vinyl stearate, dimethyl allyl propionate/vinyl stearate, vinyl propionate/vinyl stearate, cross-linked with 0.2% of divinylbenzene, vinyl dimethyl propionate/vinyl laurate, cross-linked with 0.2% of divinylbenzene, vinyl acetate/octadecyl vinyl ether, cross-linked with 0.2% de tetraallyloxyethane, vinyl acetate/allyl stearate, cross-linked with 0.2% of divinylbenzene, vinyl acetate/octadecenel cross-linked with 0.2% of divinylbenzene and allyl propionate/allyl stearate cross-linked with 0.2% of divinylbenzene.

Examples of liposoluble film-forming polymers that may also be mentioned are liposoluble copolymers, in particular those resulting from copolymerization of vinyl esters containing 9 to 22 carbon atoms or of acrylates or of alkyl methacrylates, the alkyl radicals containing 10 to 20 carbon atoms.

Such liposoluble copolymers may be selected from copolymers of polyvinyl stearate, polyvinyl stearate cross-linked with the aid of divinylbenzene, diallylether or diallyl phthalate, copolymers of stearyl poly(meth)acrylate, polyvinyl laurate, lauryl poly(meth)acrylate; these poly(meth)acrylates may be cross-linked with the aid of ethylene glycol dimethacrylate or tetraethylene glycol.

The liposoluble copolymers defined above are known and in particular described in application FR-A-2 232 303; they may have a mass average molecular weight lying in the range 2000 to 500,000, and preferably in the range 4000 to 200,000.

Examples of liposoluble film-forming polymers that may be used in the invention and that may also be mentioned are polyalkylenes and in particular copolymers of C₂-C₂₀ alkenes such as polybutene, alkylcelluloses with a C₁ to C₈, linear or branched alkyl radical, saturated or unsaturated, such as ethylcellulose or propylcellulose, copolymers of vinylpyrrolidone (VP) and in particular copolymers of vinylpyrrolidone and C₂ to C₄₀ alkene, more preferably C₃ to C₂₀. Examples of VP copolymers that may be used in the invention and that may be mentioned are the copolymer of VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene and VP/acrylic acid/lauryl methacrylate.

The lipophilic film-forming polymer may be selected from vinyl acetate/allyl stearate copolymer (in particular sold under the name Mexomere PQ by Chimex), polyvinyl laurate (in particular sold under the name Mexomere PP by Chimex), vinylpyrrolidone eicosene copolymer (in particular sold under the name Antaron V 220 by ISP, and mixtures thereof.

Silicone resins may also be mentioned, generally soluble or swellable in silicone oils, which are cross-linked polyorganosiloxane polymers. The nomenclature of silicone resins is known under the name “MDTQ”, the resin being described as a function of the various monomeric siloxane units it includes, each of the letters “MDTQ” characterizing one type of unit.

Examples of commercially available polymethylsilsesquioxane resins that may be mentioned are those that are sold by the supplier Wacker under the reference Resin MK such as Belsil PMS MK, and by the supplier SHIN-ETSU under the references KR-220L.

Examples of commercially available polypropylsilsesquioxane resins that may be mentioned are those that are sold under the reference DC670 by the supplier Dow Corning.

Siloxysilicate resins that may be mentioned are trimethylsiloxysilicate resins (TMS) such as those sold under the reference SR1000 by the supplier General Electric or under the reference TMS 803 by the supplier Wacker. It is also possible to mention trimethylsiloxysilicate resins sold in a solvent such as cyclomethicone, sold under the name “KF-7312J” by the supplier Shin-Etsu, or “DC 749”, “DC 593” by the supplier Dow Corning.

It is also possible to mention silicone resin copolymers such as those mentioned above with the polydimethylsiloxanes, such as the pressure-sensitive adhesive copolymers sold in particular by the supplier Dow Corning under the reference BIO-PSA and described in the document U.S. Pat. No. 5,162,410, or silicone copolymers derived from the reaction of a silicone resin such as those described above and of a diorganosiloxane such as that described in the document WO 2004/073626.

The lipophilic film-forming polymer may also be a vinyl polymer including at least one motif derived from carbosiloxane dendrimer.

Vinyl polymers including motifs derived from carbosiloxane dendrimer, such as, for example, those described in documents WO 2006/058793 and EP 1 862 162, are also suitable for use in the invention.

In particular, the vinyl polymer may have a skeleton and at least one lateral chain that comprises a carbosiloxane dendrimer structure. In the context of the present invention, the term “carbosiloxane dendrimer structure” represents a molecular structure having branched groups having high molecular weights, said structure having a high regularity in the radial direction starting from the bond to the skeleton. Such carbosiloxane dendrimer structures are described in the form of a highly branched siloxane-silylalkylene copolymer in the laid-open Japanese patent application Kokai 9-171 154.

Vinyl polymers grafted with at least one motif derived from carbosiloxane dendrimer that are particularly suitable for the present invention are the polymers sold under the names TIB 4-100, TIB 4-101, TIB 4-120, TIB 4-130, TIB 4-200, FA 4002 ID (TIB 4-202), TIB 4-220, and FA 4001 CM (TIB 4-230) by the supplier Dow Corning.

It is also possible to use silicone polyamides of the polyorganosiloxane type such as those described in documents U.S. Pat. No. 5,874,069, U.S. Pat. No. 5,919,441, U.S. Pat. No. 6,051,216, and U.S. Pat. No. 5,981,680.

These silicone polymers may belong to the following two families:
polyorganosiloxanes comprising at least two groups capable of establishing hydrogen interactions, these two groups being located in the polymer chain; and/or
polyorganosiloxanes comprising at least two groups capable of establishing hydrogen interactions, these two groups being located on grafts or branches.

Examples of liposoluble film-forming polymers that may be used in the invention and that may also be mentioned are polyalkylenes and in particular copolymers of C₂-C₂₀ alkenes such as polybutene, alkylcelluloses with a C₁ to C₈, linear or branched alkyl radical, saturated or unsaturated, such as ethylcellulose or propylcellulose.

In exemplary embodiments of the invention, the film-forming polymer is a linear film-forming ethylenic block polymer that preferably comprises at least one first block and at least one second block having different glass transition temperatures (Tg), said first and second blocks being connected together via an intermediate block comprising at least one constituent monomer of the first block and at least one constituent monomer of the second block.

Advantageously, the first and second blocks of block polymer are incompatible with each other.

Such polymers are described in documents EP 1 411 069 and WO 04/028488, for example.

Other examples of film-forming systems that may be used in the compositions of the invention and that may be mentioned are systems in which the film is formed in-situ on application of the composition or of a mixture of compositions containing two silicone compounds that react when they are put into contact with each other. Such systems are described in particular in application WO 2007/071706. Systems of that type are also described in applications US 2007/142575 and US 2007/142599.

As described above, in a composition of the invention, the liposoluble film-forming polymer is present at a content of active material that is greater than or equal to 9% by weight relative to the total weight of the composition.

In particular, it may be present at a content of active material lying in the range 10% to 30% by weight relative to the total weight of said composition, e.g. greater than or equal to 12% by weight, e.g. lying in the range 12% to 25% by weight.

In a preferred variant embodiment, a composition of the invention combines at least 30% to 40% by weight of isododecane, 5% to 20% by weight of at least one oil of volatility that is less than that of isododecane, preferably hydrocarbon oil, and 9% to 20% by weight of at least one indene resin expressed by weight of active material.

### Particulate Phase

The composition of the invention also includes at least one particulate phase containing at least one compound selected from pigments, nacres, fillers, and waxes.

In particular, the particulate phase consists exclusively of pigments, nacres, fillers, and/or waxes.

In the meaning of the invention, the expression “particulate phase” does not cover hydrophilic or lipophilic gelling agents, like modified clays, e.g. those known as bentone clays.

The particulate phase may in particular represent at least 20% by weight relative to the total weight of the composition, e.g. at least 25% by weight.

### Waxes

Wax may be present in the composition at a content lying in the range 0.1% to 50% by weight relative to the total weight of the composition, preferably lying in the range 1% to 25% by weight, and preferably lying in the range 5% to 15% by weight.

In preferred exemplary embodiments, the content by weight of pigments, nacres, and fillers may be greater than the content by weight of waxes.

In the meaning of the present invention, the term waxes means a lipophilic compound, solid at ambient temperature (25° C.), with a reversible solid/liquid change of state, with a melting point that is greater than or equal to 30° C. and up to 120° C.

The melting point of the wax may be measured by means of a differential scanning calorimeter (DSC), for example the calorimeter sold under the name DSC 30 by the supplier METLER.

The waxes may be hydrocarbon, fluorinated, and/or silicone waxes, and may be of vegetable, mineral, animal, and/or synthetic origin. In particular, the waxes have a melting point that is greater than 25° C., and better greater than 45° C.

In particular, it is possible to use hydrocarbon waxes such as beeswax, lanolin wax, and China insect waxes; rice-bran wax, carnauba wax, candelilla wax, ouricurry wax, alfa wax, cork fiber wax, sugar cane wax, Japan wax, and sumac wax; montan wax, microcrystalline waxes, paraffin waxes, and ozokerite waxes; polyethylene waxes, waxes obtained by Fisher and Tropsch synthesis, and waxy copolymers and esters thereof.

Mention may also be made of waxes obtained by catalytic hydrogenation of animal or vegetable oils having linear or branched C₈-C₃₂ fatty chains.

In particular, amongst said waxes mention may be made of hydrogenated jojoba oil, isomerized jojoba oil such as transisomerized partially hydrogenated jojoba it manufactured or sold by the supplier Desert Whale under the trade reference ISO-JOJOBA-500, hydrogenated sunflower oil, hydrogenated castor oil, hydrogenated coconut oil, hydrogenated lanolin oil, di-(1,1,1-trimethylolpropane) tetrastearate, sold under the name HEST 2T-4S by the supplier HETERENE, di-(1,1,1-trimethylolpropane) tetrabehenate, sold under the name HEST 2T-4B by the supplier HETERENE.

Mention may also be made of silicone waxes such as alkyl or alkoxy dimethicone containing 16 to 45 carbon atoms, and fluorinated waxes.

It is also possible to use the wax obtained by hydrogenation of olive oil esterified with stearyl alcohol, sold under the name PHYTOWAX Olive 18 L 57, or even the waxes obtained by hydrogenation of castor oil esterified with cetyl alcohol, sold under the names PHYTOWAX ricin 16L64 and 22L73 by the supplier SOPHIM. Such waxes are described in application FR-A-2792190.

The composition may include at least one polar wax.

The term “polar wax” means waxes including in their chemical structure, in addition to carbon and hydrogen atoms, at least one strongly electronegative heteroatom such as O, N, or P.

Preferably, the wax is selected from carnauba wax, candelilla wax, natural (or bleached) beeswax, and synthetic beeswax. An example of a synthetic beeswax that may be mentioned is the wax sold under the name Cyclochem 326 A by Evonik Goldschmidt (INCI name: Synthetic Beeswax).

The composition may include at least one wax presenting hardness lying in the range 0.05 MPa to 15 Mpa, and preferably lying in the range 6 Mpa to 15 MPa.

Hardness is determined by measuring the compression force measured at 20° C. by means of the texturometer that is sold under the name TA-TX2i by the supplier RHEO, and that is fitted with a stainless steel cylinder having a diameter of 2 mm that moves at the measurement speed of 0.1 millimeters per second (mm/s), and that penetrates into the wax to a penetration depth of 0.3 mm.

### Fillers

The fillers may be present in the composition at a content lying in the range 0.01% to 50% by weight relative to the total weight of the composition, preferably lying in the range 0.1% to 30% by weight, e.g. lying in the range 1% to 25% by weight, e.g. lying in the range 5% to 20% by weight.

The term “fillers” means particles of any shape, colorless or white, inorganic or synthetic, and insoluble in the composition medium regardless of the temperature at which the composition is manufactured, to the exclusion of fibers. These fillers serve in particular to modify the rheology or the texture of the composition.

The fillers may be organic or inorganic and of any shape, flakes, spherical, or oblong, whatever the crystal shape (e.g. sheets, cubic, hexagonal, orthorombic, etc).

Mention may be made of mica, kaolin, poly-β-alanine and polyethylene powders, tetrafluoroethylene polymer powders (Teflon®), lauroyl-lysine, starch, boron nitride, hollow polymeric microspheres such as those formed from polyvinylidene chloride/acrylonitrile, such as Expancel®(Nobel Industrie), acrylic acid copolymers (Polytrap® from the supplier Dow Corning) and silicone resin microbeads (e.g. Tospearls® from the supplier Toshiba), particles of polyorganosiloxane elastomers, precipitated calcium carbonate, magnesium carbonate and bicarbonate, hydroxyapatite, hollow silica microspheres (Silica Beads® from the supplier Maprecos), glass or ceramic microcapsules, metallic salts derived from organic carboxylic acids containing 8 to 22 carbon atoms, preferably 12 to 18 carbon atoms, for example zinc, magnesium, or lithium stearate, zinc laurate, and magnesium myristate.

In particular, they may preferably be selected from talc, silica, rice starch, polyamide powders (Nylon®), and polymethylmethacrylate powders, in particular those sold under the name COVABEAD® LH85 by the supplier LCW.

### Pigments and Nacres

The pigments and the nacres may be present in the composition at a content lying in the range 0.01% to 50% by weight relative to the total weight of the composition, e.g. lying in the range 0.1% to 30% by weight, e.g. lying in the range 1% to 20% by weight, e.g. lying in the range 3% to 10% by weight.

The pigments may be white or colored, inorganic and/or organic, and optionally coated.

Among inorganic pigments, mention may be made of titanium dioxide that is optionally surface treated, zirconium, zinc, or cerium oxides, together with iron or chromium oxides, manganese violet, ultramarine, chromium hydrate, and iron blue.

Among organic pigments, mention may be made of carbon black, D&C type pigments, lakes based on cochineal carmine, barium, strontium, calcium, or aluminum.

The term “nacre” or “nacre pigments” means colored particles of any form, that may optionally be iridescent, as produced in the shells of certain mollusks, or that are synthesized, and that exhibit a “pearlescent” coloring effect by optical interference.

The nacres may be selected from white nacre pigments such as mica coated with titanium or bismuth oxychloride, colored nacre pigments such as mica titanium with iron oxides, mica titanium with ferric blue or chromium oxide, mica titanium with an organic pigment of the type mentioned above, as well as nacre pigments based on bismuth oxychloride.

In particular exemplary embodiments, iron oxides are present at a content lying in the range 0.01% to 15% by weight relative to the total weight of said composition, and preferably in the range 0.01% to 10% by weight.

In preferred exemplary embodiments of the invention, the ratio by weight between the liposoluble film-forming polymer and the particulate phase may lie in the range 0.25 to 1, e.g. in the range 0.33 to 0.75, e.g. in the range 0.5 to 0.67.

In other preferred exemplary embodiments of the invention, the content by weight of the oil of volatility that is less than that of isododecane may be less than the content by weight of the particulate phase.

### Additives

The composition of the invention may further include any additive that is usually used in cosmetics.

Naturally, the person skilled in the art takes care to select any complementary cosmetic additives and/or their quantities in such a manner that the advantageous properties of the composition of the invention is spoilt little, if at all, by the envisaged addition.

Examples of particular additives that may be mentioned are antioxidants, preservatives, fragrances, neutralizers, thickeners, vitamins, fibers, and mixtures thereof.

The term “fiber” means an object of a length L and of a diameter D such that L is greater than D, and preferably much greater than D, D being the diameter of the circle in which the section of the fiber is inscribed. In particular, the ratio L/D (or form factor) is selected so as to be in the range 3.5 to 2500, preferably in the range 5 to 500, and better in the range 5 to 150.

The fibers used in the composition of the invention may be fibers of synthetic or natural, mineral or organic origin. They may be short or long, unitary or organized, for example braided, hollow or solid. They may have any shape, in particular a circular or polygonal section (square, hexagonal or octagonal) depending on the specific application envisaged. In particular, their ends are blunted and/or polished to avoid injury.

In particular, the fibers have a length lying in the range 1 μm to 10 mm, preferably in the range 0.1 mm to 5 mm, and better in the range 0.3 mm to 3 mm. Their section may be comprised in a circle with a diameter lying in the range 2 nanometers (nm) to 500 μm, preferably lying in the range 100 nm to 100 μm, and better in the range 1 μm to 50 μm. The weight or fineness of the fibers is usually expressed in denier or decitex and represents the weight in grams of 9 km of filament. Preferably, the fibers of the invention have a weight selected from the range 0.01 to 10 denier, preferably in the range 0.1 to 2 denier, and better in the range 0.3 to 0.7 denier.

Fibers that may be used in the compositions of the invention may be selected from rigid or non-rigid fibers, they may be of synthetic or natural, organic or inorganic, origin.

Furthermore, the fibers may optionally be surface treated, coated or non-coated, colored or colorless.

Examples of fibers that may be used in the compositions of the invention and that may be mentioned are non-rigid fibers such as polyamide fibers (Nylon®) or rigid fibers such as polyimide-amide fibers, such as those sold under the names KERMEL®, KERMEL TECH® by the supplier RHODIA or poly-(p-phenylene-terephthalamide) (or aramide) sold in particular under the name Kevlar® by the supplier DUPONT DE NEMOURS.

The fibers may be present at a content lying in the range 0.01% to 10% by weight relative to the total weight of the composition, in particular in the range 0.1% to 5% by weight, and more particularly in the range 0.3% to 3% by weight.

The compositions of the invention may also include at least one coloring substance selected from liposoluble colorants and hydrosoluble colorants.

Examples of liposoluble colorants are Sudan red, D&C Red 17, D&C Green 6, β-carotene, soya oil, Sudan brown. D&C Yellow 11, D&C Violet 2, D&C orange 5, quinoline yellow, and annatto.

The coloring substances may be present at a content lying in the range 0.01% to 30% by weight relative to the total weight of the composition.

The composition of the invention may be manufactured by known methods, generally used in the field of cosmetics.

The invention is illustrated in greater detail in the following example that is presented by way of non-limiting illustration of the invention.

### EXAMPLE 1

The mascara composition is made as follows.

| | |
|---|---|
| Styrene/methyl styrene/indene hydrogen copolymer | 10.00%* |
| (REGALITE R1100 HYDROCARBON RESIN | |
| P75269LO ® (Eastman Chemical) | |
| Candelilla wax (CANDELILLA WAX SP 75 G - STRAHL | 2.80% |
| & PITSCH) | |
| Ceresin (CERESIN WAX SP 254P - STRAHL & PITSCH) | 2.00% |
| Dextrine palmitate (RHEOPEARL TL2-OR - CHIBA | 2.00% |
| FLOUR MILLING) | |
| Dextrine palmitate (RHEOPEARL KL2-OR - CHIBA | 0.50% |
| FLOUR MILLING) | |
| Carnauba wax (CARNAUBA WAX SP 63 - STRAHL & | 2.00% |
| PITSCH) | |
| Polyethylene wax (PERFORMALENE 655 | 2.00% |
| POLYETHYLENE - NEW PHASE TECHNOLOGIES) | |
| Black iron oxide (SUNPURO BLACK IRON OXIDE | 7.00% |
| C33-7001 - SUN) | |
| Disteardimonium hectorite (BENTONE 38 VCG - | 4.00% |
| ELEMENTIS) | |
| Talc (J 68 BC - US COSMETICS (MIYOSHI)) | 5.00% |
| Dehydroacetic acid (11194 DEHYDROACETIC ACID | 0.50% |
| 98% - ACROS ORGANICS) | |
| Acrylate/dimethicone copolymer (KP-545 - SHIN ETSU) | 0.90% |
| Trimethylsiloxysilicate (KF - 7312 J - SHIN ETSU) | 3.20% |
| Isododecane (INEOS) | 38.35% |
| Isohexadecane (INEOS) | 12.95% |
| Propylene carbonate (JEFFSOL PROPYLENE | 0.80% |
| CARBONATE - HUNSTMAN) | |
| 2 mm aramid fibers (KERMEL TECH/2,2 | 3.00% |
| DTEX/2MM/SECHEE/VENTILEE/IRRADIEE - KERMEL) | |
| Hydrophilic silica (AEROSIL 200 - EVONIK DEGUSSA) | 3.00% |

*corresponding to 9.95% by weight of active material.

### Resistance to Friction or Rubbing:

In the present application, the term “resistance to friction” means the in vitro resistance to friction evaluated using the following protocol:
the composition is applied to three Caucasian straight hair specimens each containing thirty knots (sixty 1 cm long eyelashes) forming a 2 cm long fringe, by performing three×ten passes that are spaced apart by 2 minutes, with composition being taken between each series of ten. Each specimen is then dried at ambient temperature for a drying time of one hour.

The made up specimen is then positioned perpendicularly above a sheet of paper and rubbed by means of a hard brush of the Keracils® type (thirty passes). The quantity of grains formed in this way and collected on the sheep of paper are evaluated.
A score lying in the range 0 to 6 is attributed to the quantity of grains; 0 being the score for which no grains are collected on the sheet of paper, and 6 being the score for which a very large quantity of grains are collected.

The mascara composition of the invention presents very good resistance to friction (score=1).

1. A cosmetic composition for making up and/or caring for keratinous fibers, said composition comprising at least:
a) an oily phase formed at least of:
isododecane; and
at least 4% by weight, relative to the total weight of the composition, of at least one oil of volatility that is less than that of isododecane;
b) at least 9% by weight, relative to the total weight of the composition, of active material of at least one liposoluble film-forming polymer; and
c) a particulate phase containing at least one compound selected from pigments, nacres, fillers, and waxes, and representing at least 15% by weight of the total weight of the composition;
said composition including at least 20% by weight of isododecane relative to its total weight;
the content by weight of the oil of volatility that is less than that of isododecane being less than the content by weight of the particulate phase.

2. A composition according to claim 1, wherein the particulate phase represents at least 20% by weight relative to the total weight of the composition.

3. A composition according to claim 1, including at least 25% by weight of isododecane relative to its total weight.

4. A composition according to claim 1, wherein the oil of volatility that is less than that of the isododecane is present at a content that is greater than or equal to 5% by weight relative to the total weight of the composition.

5. A composition according to claim 1, wherein the oil of volatility that is less than that of isododecane is an oil selected from linear or branched volatile hydrocarbon oils having at least 13 carbon atoms.

6. A composition according to claim 1, wherein the oil of volatility that is less than that of isododecane is selected from hydrogenated polyisobutylene, isohexadecane, and mixtures thereof.

7. A composition according to claim 1, wherein the liposoluble film-forming polymer is present at a content of active material that is greater than or equal to 12% by weight relative to the total weight of the composition.

8. A composition according to claim 1, wherein the liposoluble film-forming polymer is a hydrocarbon polymer.

9. A composition according to claim 1, wherein the content by weight of isododecane is greater than the content by weight of the oil of volatility that is less than that of isododecane.

10. A composition according to claim 1, wherein the ratio by weight between the liposoluble film-forming polymer and the particulate phase lies in the range 0.25 to 1.

11. A composition according to claim 1, wherein the content by weight of pigments, nacres, and fillers is greater than the content by weight of waxes.

12. A composition according to claim 1, wherein the composition is a mascara.

13. A composition according to claim 1, wherein the oily phase of the composition comprises, in addition to isododecane and an oil of volatility that is less than that of isododecane, a second oil of volatility that is less than that of isododecane.

14. A composition according to claim 13, wherein said second oil of volatility that is less than that of isododecane represents less than 10% by weight of the total weight of the composition or said composition is even exempted of said oil.

15. A method of making up and/or caring for keratinous fibers, said method comprising at least applying at least one composition as defined according to claim 1 on said keratinous fibers.
